(19) United States
(12) Patent Application Publication
(10) Pub. No.: US 2022/0072051 A1
(43) Pub. Date: Mar. 10, 2022

(54) PROPHYLACTIC AND/OR THERAPEUTIC AGENT FOR DISEASES ACCOMPANIED BY FIBROSIS

(71) Applicant: PUBLIC UNIVERSITY CORPORATION YOKOHAMA CITY UNIVERSITY, Kanagawa (JP)

(72) Inventors: Hideki TANIGUCHI, Kanagawa (JP); Soichiro MURATA, Kanagawa (JP)

(73) Assignee: PUBLIC UNIVERSITY CORPORATION YOKOHAMA CITY UNIVERSITY, Kanagawa (JP)

(21) Appl. No.: 17/421,129

(22) PCT Filed: Jan. 6, 2020
(86) PCT No.: PCT/JP2020/000043

§ 371 Date: Jul. 7, 2021

(30) Foreign Application Priority Data
Jan. 9, 2019 (JP) 2019-001578

(51) Int. Cl. A61K 35/28; A61K 35/44; A61K 35/407; A61P 1/16

(52) U.S. Cl. CPC A61K 35/28; A61P 1/16; A61K 35/407; A61K 35/44

(57) ABSTRACT

The present invention provides a prophylactic and/or therapeutic agent for diseases accompanied by fibrosis. A pharmaceutical composition for preventing and/or treating a disease accompanied by fibrosis in an organ and/or a tissue, which comprises a cell mixture and/or a cell condensate comprising mesenchymal cells and vascular cells (and hepatocytes, optionally). An agent capable of inhibiting organ and/or tissue fibrosis, which comprises both mesenchymal cells and vascular cells or a cell condensate thereof. By transplanting into a subject a cell mixture and/or a cell condensate comprising mesenchymal cells and vascular cells (and optionally, hepatocytes), expression levels of fibrolysis enzymes (fibrolytic factors) such as MMP1 or MMP13 are elevated, which eventually enables inhibition of fibrosis in an organ and/or a tissue, as well as prevention and/or treatment of a disease accompanied by fibrosis in an organ and/or a tissue.

### TECHNICAL FIELD

The present invention relates to a prophylactic and/or therapeutic agent for diseases accompanied by fibrosis.

### BACKGROUND ART

Liver cirrhosis is the terminal condition of various liver diseases and causes remarkable fibrosis in the liver. Although liver transplantation is the only definitive treatment for late-stage liver cirrhosis, donor shortage is predominantly severe.

Mesenchymal stem cells, which are tissue stem cells, are considered to have a capacity to differentiate into osteoblasts, adipocytes, myocytes, chondrocytes, etc. that are belonging to the mesenchymal lineage. Therefore, application of mesenchymal stem cells to regenerative medicine is expected. Further, mesenchymal stem cells have been found to have an immunoinhibitory effect and are greatly expected as an agent for cell-based therapy for treatment-resistant immune diseases. Mesenchymal stem cells may be either collected from somatic cells (bone marrow or adipose tissue) or induced from human iPS cells by directed differentiation. A number of clinical researches have been made on cell-based therapy using mesenchymal stem cells. As a method of transplantation, a technique is generally used in which mesenchymal stem cells are administered to the peripheral vein, the portal vein, etc. in the state of a single cell. The effect of such transplantation is temporary, and there have been no reports that histologically transplanted mesenchymal stem cells differentiated into hepatocytes or long term engraftment thereof could be confirmed. Cell sheets of mesenchymal stem cells have been released, but no clear effect on liver diseases has been reported.

Vascular endothelial cells are cells coating the intravascular space, and are playing the central role in vascular function. In researches using human vascular endothelial cells, umbilical vein derived vascular endothelial cells and cells induced from pluripotent stem cells such as human iPS cells by directed differentiation are used (Japanese Patent No. 5920741 (Patent Document No. 1), etc.). Further, a method of preparing an iPS cell-derived vascular progenitor cell sheet has also been reported (WO 2013/069661 (Patent Document No. 2)). However, no therapeutic effect on liver diseases has been reported in vascular endothelial cells, vascular progenitor cell sheets, etc.

Since mesenchymal stem cells and vascular endothelial cells are considered as supporting tissues for forming organs and tissues, medical efficacy of these cells or cell condensates composed of these cells alone has not been reported. In particular, effect of these cells or cell condensates thereof on improvement of fibrosis in an organ or tissue, such as liver, has not been reported.

To date, the present inventors have succeeded in creating an organ bud by coculture of hepatic endodermal cells of an optimum differentiation stage obtained from pluripotent stem cells such as iPS cells with vascular endothelial cells and mesenchymal cells, in which these three different cell components are cultured at an optimal mixing ratio (Patent Document No. 3). Further, it is possible to provide an in vitro prepared biological tissue with a vascular system by coculturing vascular cells and mesenchymal cells (Patent Document No. 4). The present inventors have also succeeded in developing a technique for preparing spheroids of uniform size highly efficiently (Patent Document No. 5). However, in these conventional techniques, long term engraftment and therapeutic effect on liver cirrhosis have not been reported for cell condensates composed of pluripotent stem cells.

### PRIOR ART LITERATURE

### Patent Documents

Patent Document No. 1: Japanese Patent No. 5920741

Patent Document No. 2: WO 2013/069661

Patent Document No. 3: WO 2013/047639 A1

Patent Document No. 4: WO 2015/012158 A1

Patent Document No. 5: WO 2014/196204 A1

### DISCLOSURE OF THE INVENTION

### Problem for Solution by the Invention

It is an object of the present invention to provide a prophylactic and/or therapeutic agent for diseases accompanied by fibrosis.

### Means to Solve the Problem

To date, the present inventors have developed a method of transplanting fetal liver tissue on the surface of a liver cirrhosis model animal. By the fetal liver tissue transplantation, engraftment of liver tissue, improvement of liver function, improvement of fibrosis and improvement of survival rate were observed (The Japanese Society for Regenerative Medicine, 2018). It is believed that fetal liver tissue has a structure resembling the structure of a cell condensate (liver bud) composed of three types of cells induced from pluripotent stem cells by directed differentiation (i.e., hepatic endodermal cells, mesenchymal stem cells and vascular endothelial cells). Using these basic techniques, a cell condensate composed of these three types of cells induced from pluripotent stem cells by directed differentiation was transplanted into a liver cirrhosis model animal. As a result, improvement of liver fibrosis and improvement of survival rate by pluripotent stem cells were observed; these effects had been difficult to achieve by conventional techniques. Cells which mainly contribute to the improvement of fibrosis turned out to be mesenchymal stem cells and vascular endothelial cells as a result of analyses such as microarray analysis, single cell RNA sequence analysis, immunostaining, etc. When these two types of cells were cultured in mixture, expression of matrix metalloproteinases (MMPs) was elevated remarkably compared to the case where these cells were cultured alone. Fibrosis is caused by production of collagen by stellate cells in the liver upon activation by TGF β stimulation. In cell condensates composed of two types of cells (hereinafter, “two-type cell condensates”), TGF β-inhibitory factors such as decorin were highly expressed. From what have been described so far, it is believed that even transplantation of two-type cell condensates of mesenchymal stem cells and vascular endothelial cells is capable of improving fibrosis in an organ or tissue, such as liver. Actually, when the two-type cell condensates were transplanted into liver cirrhosis model animals, improvement of liver fibrosis and improvement of survival rate were observed.

The gist of the present invention is as follows.
(1) A pharmaceutical composition for preventing and/or treating a disease accompanied by fibrosis in an organ and/or a tissue, which comprises a cell mixture and/or a cell condensate comprising mesenchymal cells and vascular cells.
(2) The composition of (1) above, wherein the ratio of mesenchymal cells to vascular cells in the cell mixture and/or the cell condensate is 1-10:10-1.
(3) The composition of (1) or (2) above, wherein the cell condensate comprising mesenchymal cells and vascular cells has been prepared by coculturing mesenchymal cells and vascular cells.
(4) The composition of any one of (1) to (3) above, wherein the mesenchymal cell is undifferentiated.
(5) The composition of any one of (1) to (4) above, wherein the vascular cell is vascular endothelial cells.
(6) The composition of any one of (1) to (5) above, wherein the mesenchymal cell is derived from ES cells or iPS cells.
(7) The composition of any one of (1) to (6) above, wherein the vascular cell is derived from ES cells or iPS cells.
(8) The composition of any one of (1) to (7) above, wherein the cell mixture and/or the cell condensate further comprises hepatocytes.
(9) The composition of (8) above, wherein the ratio of hepatocytes, mesenchymal cells and vascular cells in the cell mixture and/or the cell condensate is 10:0.1-10:0.1-10.
(10) The composition of (8) or (9) above, wherein the cell condensate comprising hepatocytes, mesenchymal cells and vascular cells has been prepared by coculturing hepatocytes, mesenchymal cells and vascular cells.
(11) The composition of any one of (8) to (10) above, wherein the hepatocyte is hepatic endodermal cells.
(12) The composition of any one of (8) to (11) above, wherein the hepatocyte is derived from ES cells or iPS cells.
(13) The composition of any one of (1) to (12) above, which is to be transplanted onto the surface of an organ and/or a tissue where fibrosis has occurred.
(14) An agent capable of inhibiting organ and/or tissue fibrosis, which comprises a cell mixture and/or a cell condensate comprising mesenchymal cells and vascular cells.
(15) The agent of (14) above, wherein the cell mixture and/or the cell condensate further comprises hepatocytes.
(16) A method of preventing and/or treating a disease accompanied by fibrosis in an organ and/or a tissue, which comprises transplanting into a subject a pharmaceutically effective amount of a cell mixture and/or a cell condensate comprising mesenchymal cells and vascular cells.
(17) The method of (16) above, wherein the cell mixture and/or the cell condensate further comprises hepatocytes.
(18) A method of inhibiting fibrosis in an organ and/or a tissue, which comprises transplanting into a subject a pharmaceutically effective amount of a cell mixture and/or a cell condensate comprising mesenchymal cells and vascular cells.
(19) The method of (18) above, wherein the cell mixture and/or the cell condensate further comprises hepatocytes.
(20) Use of a cell mixture and/or a cell condensate comprising mesenchymal cells and vascular cells for prevention and/or treatment of a disease accompanied by fibrosis in an organ and/or a tissue.
(21) The use of (20) above, wherein the cell mixture and/or the cell condensate further comprises hepatocytes.
(22) Use of a cell mixture and/or a cell condensate comprising mesenchymal cells and vascular cells, the use being in a method of preventing and/or treating a disease accompanied by fibrosis in an organ and/or a tissue.
(23) The use of (22) above, wherein the cell mixture and/or the cell condensate further comprises hepatocytes.
(24) Use of a cell mixture and/or a cell condensate comprising mesenchymal cells and vascular cells for inhibiting fibrosis in an organ and/or a tissue.
(25) The use of (24) above, wherein the cell mixture and/or the cell condensate further comprises hepatocytes.

### Effect of the Invention

According to the present invention, it is possible to improve fibrosis in an organ or tissue, such as liver.

The present specification encompasses the contents of the specification and/or drawings disclosed in Japanese Patent Application No. 2019-1578 based on which the present application claims priority.

### BRIEF DESCRIPTION OF THE DRAWINGS

FIG. 1 shows two-type cell condensate and three-type cell condensate. Upper panel, left: an image of three-type cell condensate (liver bud) (hepatic endodermal cells:vascular cells:mesenchymal cells=10:7:1). Upper panel, right: images of two-type cell condensates (vascular cells:mesenchymal cells=7:1, 1:1 and 1:7). Equivalent morphologies are recognized. Upper row shows cell condensates prepared with micropattern plates. Bottom row shows cell condensates prepared with hydrogel. Vascular cells are stained with Kusabira Orange. Lower panel: images of fused three-type cell condensates (hepatic endodermal cells:vascular cells:mesenchymal cells=10:7:1, 10:7:7, 10:4:4 and 10:2:2). Small-size three-type cell condensates prepared on micropattern plates were fused on cell culture inserts (at day 0 and day 9 of fusion).

FIG. 2 Comprehensive gene expression analysis was performed on hepatic endodermal cells (grouped by differentiation stage: DE, HE, IH and MET), three-type cell condensates, two-type cell condensates, hepatocytes (Liver) and bone marrow-derived mesenchymal stem cells. As a result, expression levels of fibrolytic factors such as MMP2, MMP3, MMPI, etc., matrix synthesis inhibitory factors such as decorin, TRAIL, etc., MIF (macrophage directed induction) and so forth were elevated in both two-type and three-type cell condensates.

FIG. 3 Cytokine array analysis was performed on culture supernatants of three-type cell condensates. The results revealed that production levels of cytokines such as Emmprin (MMP induction), HGF (inhibition of fibrosis in stellate cells), FGF19, MMP9, DKK1 (these three inhibit fibrosis), CXCL1 (M1 macrophage induction), IL4 (M2 macrophage induction), CCL20 (immunocyte recruitment), MIF (macrophage recruitment in the liver), GDF15 (inhibition of immunocyte function), etc. were elevated.

FIG. 4 The results of single cell RNA sequence analysis on hepatic endodermal cells, vascular cells and mesenchymal cells constituting three-type cell condensates are shown. Mouse fetal liver-constituting cells (pink), three-type cell condensate-constituting cells (red). HE: hepatic endodermal cells; EC: vascular endothelial cells; MC: mesenchymal cells. Each of the three-type cell condensate-constituting cells is expressing the gene sets of “macrophage induction & M2 macrophage polarization”, “extracellular matrix (ECM) degradation and inhibition of ECM production” and “angiogenesis”, thus contributing to improvement of fibrosis.

FIG. 5 Fused three-type or two-type cell condensate was transplanted into immunodeficient mouse liver cirrhosis model. Improvement of fibrosis two weeks after transplantation is shown by Sirius red staining. By transplantation of fused three-type or two-type cell condensate, decrease in the Sirius red-positive area was recognized.

FIG. 6 Three-type, two-type or fused three-type cell condensate was transplanted into immunodeficient rat liver cirrhosis model. Three weeks after transplantation, collagen levels in the liver tissue were compared. As regards fused three-type cell condensates, four mixing ratios were used for comparison; they were hepatic endodermal cells:vascular cells:mesenchymal cells=10:7:1, 10:7:7, 10:4:4 and 10:2:2. The liver was hydrolyzed to quantify hydroxyproline. Collagen levels in the hepatic left lobe into which cell condensates were transplanted are shown. *p<0.05, **p<0.01 vs Cirrhosis group, One way ANOVA, n=7-18.

FIG. 7 Microarray data of liver tissues from normal group, liver cirrhosis group (sham operation group) and three-type cell condensate transplantation group are shown. Decrease in fibrosis signature and increase in normalization signature were observed in three-type cell condensate transplantation group.

FIG. 8 Fused three-type cell condensates were transplanted into immunodeficient rat and mouse liver cirrhosis models. Survival rates up to 3 weeks (for rat) and up to 10 weeks (for mouse) after transplantation are shown. Left panel (rat): the survival rate of fused three-type cell condensate transplantation group was significantly improved compared to sham operation group and three-type cell condensate transplantation group. *v0.04′74. Right panel (mouse): the survival rate of fused three-type cell condensate transplantation group was significantly improved compared to sham operation group and no operation group. *p=0.0013 Log rank test.

FIG. 9 Fused three-type cell condensates were transplanted into immunodeficient rat liver cirrhosis model. Images of engrafted tissues three weeks after transplantation are shown. Human albumin-positive hepatocytes, human CD31-positive blood vessel-like structures, and human CK19-positive bile duct structures are observed.

FIG. 10 Fused three-type cell condensates were transplanted into immunodeficient mouse liver cirrhosis model. Images of macrophage accumulation and MMP9 accumulation six weeks after transplantation are shown. *p<0.05 vs sham operation group, Mann Whitney U test.

FIG. 11 Two-type and three-type cell condensates were transplanted into immunodeficient rat liver cirrhosis model. Biochemical data on the blood from 1 to 3 weeks after transplantation are shown. A tendency toward improvement was observed in hyaluronic acid, NH3, ALT and platelets as a result of transplantation. *p<0.05, **p<0.01 vs Cirrhosis group, One way ANOVA, n=3-13.

### BEST MODES FOR CARRYING OUT THE INVENTION

Hereinafter, the present invention will be described in more detail.

The present invention provides a pharmaceutical composition for preventing and/or treating a disease accompanied by fibrosis in an organ and/or a tissue, which comprises a cell mixture and/or a cell condensate comprising mesenchymal cells and vascular cells. As used herein, the term “treating” is a concept encompassing not only curing but also alleviating or improving symptoms.

The cell mixture and/or the cell condensate may further comprise hepatocytes.

In the present invention, the term “mesenchymal cell” means connective tissue cells that are mainly located in mesoderm-derived connective tissues and which form support structures for cells that function in tissues. The “mesenchymal cell” is a concept that encompasses those cells which are destined to, but are yet to, differentiate into mesenchymal cells. Mesenchymal cells used in the present invention may be either differentiated or undifferentiated. Whether a certain cell is an undifferentiated mesenchymal cell or not may be determined by examining the expression of marker proteins such as Stro-1, CD29, CD44, CD73, CD90, CD105, CD133, CD271 or Nestin (if any one or more of the above-listed marker proteins are expressed, the cell can be judged as an undifferentiated mesenchymal cell). A mesenchymal cell in which none of the above-listed markers are expressed can be judged as a differentiated mesenchymal cell. Among the terms used by those skilled in the art, the following are included in the “mesenchymal cell” of the present invention: mesenchymal stem cells, mesenchymal progenitor cells, mesenchymal cells (R. Peters, et al. PLoS One. 30; 5(12):e15689 (2010)) and so on. Mesenchymal cells may be induced from totipotent or pluripotent cells (e.g., iPS cells or ES cells) by directed differentiation. Alternatively, mesenchymal cells may be derived from somatic cells, such as bone marrow or fat tissue. Cell Reports 21, 2661-2670, 2017 discloses a method of inducing mesenchymal cells from iPS cells, and mesenchymal cells prepared by this method may be used in the present invention. The prepared mesenchymal cell (iPSC-MC) can be a cell which is CD166-positive and does not express a vascular endothelial marker CD31 (PECAM1). This cell may be septum transversum mesenchymal (STM) cell. STM cell is LHX2-positive and can be WT1-positive. As mesenchymal cells, human-derived cells are mainly used. However, mesenchymal cells derived from non-human animals (e.g., animals used, for example, as experimental animals, pet animals, working animals, race horses or fighting dogs; more specifically, mouse, rat, rabbit, pig, dog, monkey, cattle, horse, sheep, chicken, shark, devilfish, ratfish, salmon, shrimp, crab or the like) may also be used.

Although vascular cells may be isolated from vascular tissues such as umbilical veins, vascular cells are not limited to those isolated from vascular tissues and may be induced from totipotent or pluripotent cells (e.g., iPS cells or ES cells) by directed differentiation. As vascular cell, vascular endothelial cell, vascular smooth muscle cell or the like may be enumerated. Among them, vascular endothelial cell is preferable. Vascular endothelial cells derived from umbilical vein are commercially available and easy to access. In the present invention, the term “vascular endothelial cell” means cells constituting vascular endothelium or cells capable of differentiating into such cells (e.g., vascular endothelial progenitor cell, vascular endothelial stem cell, etc.). Whether a cell is vascular endothelial cell or not can be determined by examining the expression of marker proteins such as TIE2, VEGFR-1, VEGFR-2, VEGFR-3 and CD31 (if any one or more of the above-listed marker proteins are expressed, the cell can be judged as vascular endothelial cell). Further, as markers for vascular endothelial progenitor cell, c-kit, Sca-1 and the like have been reported. Whether a cell is vascular endothelial progenitor cell or not can be determined by examining the expression of these markers (S Fang, et al. PLOS Biology. 2012; 10(10):e1001407). Among the terms used by those skilled in the art, the following are included in the “vascular endothelial cell” of the present invention: endothelial cells, umbilical vein endothelial cells, endothelial progenitor cells, endothelial precursor cells, vasculogenic progenitors, hemangioblast (H J. Joo, et al. Blood. 25; 118(8):2094-104 (2011)) and so on. As used herein, the term “vascular smooth muscle cell” means cells constituting vascular smooth muscle or cells capable of differentiating into such cells (e.g., vascular smooth muscle progenitor cell, vascular smooth muscle stem cell, etc.). Vascular smooth muscle cells are commercially available. Whether a cell is vascular smooth muscle cell or not can be judged by examining the expression of alpha SMA-positive, von Will brand factor (vWF), and markers such as CD90. Cell Reports 21, 2661-2670, 2017 discloses a method of inducing vascular cells (vascular endothelial cells) from iPS cells, and vascular cells prepared by this method may be used in the present invention. The thus prepared vascular cell (iPSC-EC) is CD31-positive and can be CD144-positive. This vascular cell is convenient if the expression of at least one gene selected from the group consisting of PECAM1, CDHS, KDR and CD34 is elevated therein compared to the pluripotent stem cell before directed differentiation. As vascular cells, human-derived cells are mainly used. However, vascular cells derived from non-human animals (e.g., animals used, for example, as experimental animals, pet animals, working animals, race horses or fighting dogs; more specifically, mouse, rat, rabbit, pig, dog, monkey, cattle, horse, sheep, chicken, shark, devilfish, ratfish, salmon, shrimp, crab or the like) may also be used. Vascular cells may be obtained from umbilical cord blood, umbilical cord blood vessels, neonatal tissues, liver, aorta, brain, bone marrow, fat tissue, etc.

Hepatocyte is a concept that encompasses those cells which have been differentiated into functional cells constituting the liver or those undifferentiated cells which are capable of differentiating into such functional cells. Undifferentiated cells include stem cells, progenitor cells, endodermal cells, organ bud cells and the like. The undifferentiated cell is preferably those cells which are destined to, but are yet to, differentiate into functional cells. As “undifferentiated hepatocyte”, examples include, but are not limited to, cells capable of differentiating into endodermal organs such as liver, spleen, gastrointestinal tract (pharynx, esophagus, stomach, intestine), lung, thyroid, parathyroid, urinary tract, thymus, and so on. Whether or not a cell is capable of differentiating into an endodermal organ can be judged by examining the expression of marker proteins (if any one or more of marker proteins are expressed, the cell can be judged as a cell capable of differentiating into an endodermal organ). For example, in cells capable of differentiating into liver, HHEX, SOX2, HNF4A, AFP, ALB and the like are markers; in cells capable of differentiating into pancreas, PDX1, SOX17, SOX9 and the like are markers; in cells capable of differentiating into intestinal tract, CDX2, SOX9 and the like are markers; in cells capable of differentiating into kidney, SIX2 and SALL1 are markers; in cells capable of differentiating into heart, NKX2-5, MYH6, ACTN2, MYL7 and HPPA are markers; in cells capable of differentiating into blood, C-KIT, SCA1, TER119 and HOXB4 are markers; and in cells capable of differentiating into brain or spinal cord, HNK1, AP2, NESTIN and the like are markers. Among the terms used by those skilled in the art, the following are included in the undifferentiated hepatocyte in the present invention: hepatoblast, hepatic progenitor cells, hepatic precursor cells, and the like. Undifferentiated hepatocytes may be prepared from pluripotent stem cells (e.g., iPS cells or ES cells) according to known methods. For example, cells capable of differentiating into the liver may be prepared as previously described (K. Si-Taiyeb, et al., Hepatology, 51 (1): 297-305 (2010); T. Touboul, et al., Hepatology. 51(5):1754-65 (2010)). Examples of functional cells constituting the liver include hepatocytes of the liver. In the present invention, hepatocytes are preferably hepatic endodermal cells. Hepatic endodermal cells are prepared as follows. Briefly, SOX17-positive, CXCR4-positive embryonic endodermal cells are induced from undifferentiated iPS cells by directed differentiation, which is then advanced by one more stage. It is possible that the thus prepared hepatic endodermal cell is less than 50% in HNF4A-positive and CXCR4 positive rate, and less than 10% in Tra2-49/6E⁺ positive rate. When the hepatic endodermal cell is subjected to further directed differentiation, mature hepatocytes are obtained which secrete human albumin. Cell Reports 21, 2661-2670, 2017 discloses a method of inducing hepatocytes (hepatic endodermal cells) from iPS cells, and hepatocytes prepared according to this method may be used in the present invention. The prepared hepatocyte (iPSC-HE) is TBX3-positive and can be ADRA1B-positive. As hepatocytes, human-derived hepatocytes are mainly used. However, hepatocytes derived from non-human animals (e.g., animals used, for example, as experimental animals, pet animals, working animals, race horses or fighting dogs; more specifically, mouse, rat, rabbit, pig, dog, monkey, cattle, horse, sheep, chicken, shark, devilfish, ratfish, salmon, shrimp, crab or the like) may also be used.

The ratio of mesenchymal cells to vascular cells in a cell mixture and/or cell condensate may be 1-10:10-1. The ratio of mesenchymal cells to vascular cells (mesenchymal cells:vascular cells) is preferably set between 1:10 and 10:1. More preferably, the ratio is 1:1.

The ratio of hepatocytes, mesenchymal cells and vascular cells in a cell mixture and/or a cell condensate may be 10:0.1-10:0.1-10. The ratio of hepatocytes, mesenchymal cells and vascular cells is preferably 1-7 mesenchymal cells and 1-7 vascular cells against 10 hepatocytes. More preferably, hepatocytes:mesenchymal cells:vascular cells=10:7:1.

Mesenchymal cells and vascular cells as two types of cells may be either in a state of mixture or in a state of forming a cell condensate.

Hepatocytes, mesenchymal cells and vascular cells as three types of cells may be either in a state of mixture or in a state of forming a cell condensate.

As used herein, “cell condensate” means “condensate in which cells are adhered to each other to form a three-dimensional structure”. Preferably, the cell condensate has a strength that allows non-destructive collection. Further, cell condensates capable of interacting with each other are preferable.

It is possible to prepare a cell condensate comprising mesenchymal cells and vascular cells by coculturing mesenchymal cells and vascular cells. Further, it is possible to prepare a cell condensate comprising three types of cells, i.e., hepatocytes, mesenchymal cells and vascular cells, by coculturing hepatocytes, mesenchymal cells and vascular cells.

For example, it is possible to form a cell condensate by culturing a mixture of mesenchymal cells and vascular cells (which may further comprise hepatocytes) on a gel-like support two-dimensionally. Specifically, a support with an appropriate stiffness [e.g., a Young's modulus of 200 kPa of less (in the case of a Matrigel-coated gel of a flat shape); however, the appropriate stiffness of the support may vary depending on the coating and shape] is formed and solidified on a cell culture dish. Examples of such substrates include, but are not limited to, hydrogels (such as acrylamide gel, gelatin and Matrigel). The stiffness of the support is preferably 100 kPa or less, more preferably 1-50 kPa. The gel-like support may be planar, or alternatively, the side on which culture is to be performed may have a U- or V-shaped cross section. Further, the support may be modified by adding thereon Matrigel or laminin.

For modification, collagen, hyaluronic acid, polyethylene glycol, fibrin, etc. may also be used.

It is also possible to use a micropatterned plate for preparing cell condensates. WO 2015/182159 discloses a micropatterned plate for cell culture, and this plate may be used in the present invention.

Alternatively, cell condensates may be prepared by other methods, such as a method in which cell condensates are cultured within collagen/fibronectin gel (Transplant Proc. 2012 May; 44(4):1130-3), a hanging drop method (Journal of Visualized Experiments, 2011, 51, 1-4), etc.

The mixing ratio of two types of cells may be conveniently mesenchymal cells 1-10: vascular endothelial cells 10-1, but the ratio is not limited to this range. The mixing ratio of three types of cells may be conveniently hepatocytes 10: mesenchymal cells 0.1-10: vascular endothelial cells 0.1-10, but the ratio is not limited to this range. The culture period is preferably about 1 to 3 days, which may be changed appropriately. As a medium, a 1:1 mixture of StemPro™-34 SFM (StemPro) and Mesenchymal Stem Cell Growth Medium 2 (MSCGM2: PromoCell) supplemented with VEFG may be used, but other medium may also be used.

The temperature during culture is not particularly limited but it is preferably 30-40° C. and more preferably 37° C.

Further, cell condensates may be fused among themselves. Methods of fusing cell masses are known. Cell condensates may be fused among themselves according to any of the known cell mass fusion methods.

For example, WO 2019/189324 discloses a method of fusing cell masses, comprising seeding cell masses on a plane capable of cell adhesion and culturing the cell masses while feeding a culture medium from both the obverse and reverse sides of the cell mass-seeded plane. According to the cell mass fusion method of WO 2019/189324, it is possible to fuse cell condensates among themselves. The term “fusion among cell condensates” means that a plurality of cell condensates form a continuous structure. Cell condensates fused are capable of constructing a continuous vascular structure through internal self-organization. Fusion among cell condensates may achieve the following: the size of cell condensate is increased; a vascular network structure is formed in cell condensate; vascular network structure is developed further; and the function of cell condensate is improved. According to the cell mass fusion method of WO 2019/189324, it is possible to construct a vascular network structure in cell condensates. Moreover, it is possible to prepare a fused cell condensate of 8 mm or more in diameter. Cell condensates may be seeded on a plane capable of cell adhesion in such a manner that the ratio of the area occupied by cell condensates to the seeded plane is 40 to 100%. The ratio of the area occupied by cell condensates to the seeded plane is preferably 60 to 100% and more preferably 80 to 100%. The ratio of the area occupied by cell condensates to the seeded plane can be obtained by measuring the projected shadow area of cell condensates and calculating the ratio of this area to the area of seeded plane. The projected shadow area may be measured by the following method. Briefly, the projected shadow area of cell condensates is calculated with image analysis software such as FIJI, ImageJ, Photoshop, etc. Cell condensates may be seeded on a plane capable of cell adhesion at high density. For example, assuming a cell condensate diameter is 150 μm, high density means that the number of cell condensates present per cm³ of space may be 9.5×10⁴ to 3.8×10⁵, preferably 1.9×10⁵ to 3.8×10⁵, more preferably 2.9×10⁵ to 3.8×10⁵. The number of cell condensates may be two or more. When the number of cell condensates is increased, fused cell condensate of larger size can be obtained. The size of cell condensate is appropriately 80-500 μm, preferably 100-250 μm. The culture medium is not particularly limited. Any medium may be used as long as it is suitable for culturing cell condensates. For example, if the cell condensate is a liver bud, examples of preferable media include, but are not limited to, a medium obtained by adding dexamethasone, oncostatin M and HGF to a 1:1 mixture of EGM BulletKit (Lonza) and HCM BulletKit (Lonza) from which hEGF (recombinant human epithelial cell growth factor) has been removed; a medium which is a 1:1 mixture of EGM BulletKit (Lonza) and VascuLife EnGS Comp Kit (LCT); and a medium which is a 1:1 mixture of EGM BulletKit (Lonza) and Endothelial Cell Growth Medium MV.

It is possible to fuse cell condensates by seeding cell condensates on a plane capable of cell adhesion and culturing the cell condensates while feeding a culture medium from both the obverse and reverse sides of the cell condensate-seeded plane.

When fusion of cell condensates is performed on a plane capable of cell adhesion, if, for example, the plane capable of cell adhesion assumes the structure of a porous membrane, it would be advantageous for culturing cell condensates after fusion because nutrients can be fed to fused cell condensates from both top and bottom while allowing for efficient oxygen supply. However, the applicable plane is not limited to this embodiment. Examples of planes capable of cell adhesion include, but are not limited to, planes which have been negatively charged to acquire hydrophilicity by means of corona discharge in the atmosphere or vacuum gas plasma polymerization treatment (cell adhesion surface treatment) or the like; planes with a gelatin treated surface; planes coated with extracellular matrix (such as collagen, laminin, or fibronectin) or mucopolysaccharides (heparin sulfate, hyaluronic acid, chondroitin sulfate, etc.); planes coated with basic synthetic polymers (such as poly-D-lysine); planes with a synthetic nanofiber surface; planes with a hydrophilic and neutral hydrogel layer's surface; and collagen membrane (KOKEN CO., LTD.). In the case where the plane capable of cell adhesion assumes the structure of a porous membrane, the pore size may be 0.4-8 μm. Examples of culture equipment with a plane capable of cell adhesion include, but are not limited to, Falcon cell culture plate (Corning), Falcon multi-cell culture plate (Corning), and Falcon cell culture insert (Corning). These may be used advantageously in the present invention. Culture may be performed in any method selected from batch culture, semi-batch culture (fedbatch culture) and continuous culture (perfusion culture). Culture may be either static culture, aeration culture, spinner culture, shaking culture or rotary culture. Among these, static culture is preferable.

The temperature at the time of culture for fusing cell condensates is not particularly limited. The temperature is preferably 25-37° C.

The time period of culture for fusing cell condensates is not particularly limited. The period is preferably 1-10 days.

By fusing cell condensates, it is possible to prepare fused cell condensates with diameters of 100 μm or more, 1 mm or more, 2 mm or more, 2.5 mm or more, 4 mm or more, 6 mm or more, and 8 mm or more. Fused cell condensates with diameters of 100 μm or more, 1 mm or more, 2 mm or more, 2.5 mm or more, 4 mm or more, 6 mm or more, and 8 mm or more may be prepared from 2 to 4 cell condensates, 150-200 cell condensates, 300-400 cell condensates, 350-500 cell condensates, 600-800 cell condensates, 1200-1600 cell condensates, and 2400-2800 cell condensates, respectively, with a size of about 80-150 μm.

Further, the fused cell condensate is capable of forming a vascular network structure. In one example described later, a vascular network structure was formed in fused three-type cell condensate. As regards the vascular network structure, microvessels, arterioles/venules, sinusoids, or the like may be enumerated. Furthermore, a fused three-type cell condensate is capable of forming a bile duct structure.

Fused cell condensates may have an improved function compared to the function of cell condensates before fusion. For example, in the case where the cell mass is a liver bud, the fused liver bud can have higher gene expression levels of hepatic differentiation markers (e.g., FoxA2, AFP, CYP3A7 and CYP7A1) as compared to the liver bud before fusion. Further, the hepatic function of fused liver bud may be improved compared to that of liver buds before fusion. For example, the fused liver bud can have higher gene expression levels of hepatic differentiation markers (e.g., ALB, OTC, CYP3A7 and GLUT2) while allowing for enhanced albumin production/transferrin production and ammonium metabolism as compared to liver buds before fusion. Further, in the case where fused cell condensate is transplanted into a living body, the graft survival rate may be improved compared to that of cell condensates before fusion. Furthermore, in the case where fused cell condensate has a vascular network, anastomosis and blood perfusion between the vascular network of the fused cell condensate transplanted into a living body and the host vessels may be observed.

By transplanting a cell mixture and/or a cell condensate (of either fused type or non-fused type) comprising mesenchymal cells and vascular cells (which may further comprise hepatocytes) into a subject, expression levels of fibrolytic enzymes (fibrolytic factors) (e.g., MMP1, MMP2, MMP9 and MMP13) and cytokines such as Emmprin (MMP induction), HGF (inhibition of fibrosis in stellate cells), FGF19, MMP9, DKK1 (these three inhibit fibrosis), CXCL1 (M1 macrophage induction), IL4 (M2 macrophage induction), CCL20 (immunocyte recruitment), MIF (macrophage recruitment in the liver), GDF15 (inhibition of immunocyte function), etc. are elevated, which leads to inhibition of fibrosis in an organ and/or a tissue. Thus, the cell mixture and/or the cell condensate is capable of preventing and/or treating a disease accompanied by fibrosis in an organ and/or a tissue. Therefore, the present invention provides an agent capable of inhibiting organ and/or tissue fibrosis, which comprises a cell mixture and/or a cell condensate (of either fused type or non-fused type) comprising mesenchymal cells and vascular cells (and further comprising hepatocytes, optionally). The agent of the present invention may be used as a pharmaceutical drug or an experimental reagent. Further, the present invention also provides a method of inhibiting fibrosis in an organ and/or a tissue, comprising transplanting into a subject a pharmaceutically effective amount of a cell mixture and/or a cell condensate (of either fused type or non-fused type) comprising mesenchymal cells and vascular cells (and further comprising hepatocytes, optionally).

Fibrosis may occur in any organ in a living body, such as skin, lung, pancreas, liver, kidney, etc. Examples of diseases accompanied by fibrosis include, but are not limited to, liver cirrhosis; chronic hepatitis such as nonalcoholic steatohepatitis, alcoholic hepatitis; diseases causing fibrosis such as renal dysfunction (specifically, diabetic nephropathy, chronic glomerulonephritis), pulmonary disorder (specifically, idiopathic interstitial pneumonia, connective tissue disease-associated interstitial pneumonia, hypersensitivity pneumonitis, drug-induced pneumonia, irradiation pneumonitis, acute respiratory distress syndrome (ARDS), etc.) and skin disorder (scleroderma, decubitus, keloid, etc.).

Applicable subjects include humans and non-human animals (e.g., animals used, for example, as experimental animals, pet animals, working animals, race horses or fighting dogs; more specifically, mouse, rat, rabbit, pig, dog, monkey, cattle, horse, sheep, chicken, shark, devilfish, ratfish, salmon, shrimp, crab or the like). The site of transplantation may be any site as long as transplantation is possible. The intracranial space, the mesentery, the liver, the spleen, the kidney, the kidney subcapsular space, the supraportal space, and the like may be enumerated. However, transplantation onto the surface of an organ and/or a tissue in which fibrosis has occurred is preferable. For example, when a cell mixture and/or a cell condensate is transplanted into the liver, first, the hepatic serosa is peeled off bloodlessly and extensively using injection needles, electrosurgical knives, ultrasonic surgical aspirators, etc. To the peeled off surface of the liver, a cell mixture and/or a cell condensate (of either fused type or non-fused type) comprising mesenchymal cells and vascular cells (and further comprising hepatocytes, optionally) is transplanted. Then, the site may be fixed with a surgical covering material for clinical use. The cell mixture and/or the cell condensate may also be transplanted into organs or tissues other than liver in the same manner as described above.

A cell mixture and/or a cell condensate (of either fused type or non-fused type) comprising mesenchymal cells and vascular cells (and further comprising hepatocytes, optionally) may be transplanted (administered) to a subject in an amount effective for prevention and/or treatment, taking into consideration the age, sex, body weight, symptoms, etc. of the subject. For example, the numbers of mesenchymal cells and vascular cells (and optionally, hepatocytes) per transplantation may each range from 10⁸ to 10⁹ cells per 10 cm² of the transplantation site. Preferably, the number is 1×10⁹-2×10⁹ cells and more preferably, the number is 2×10⁹-3×10⁹ cells. The above-mentioned cell numbers are independent of whether a subject is transplanted with a cell mixture or cell condensate.

In the transplantation of a cell mixture and/or a cell condensate (of either fused type or non-fused type) comprising mesenchymal cells and vascular cells (and further comprising hepatocytes, optionally), the following may be used: medium components such as StemPro™-34 SFM, EGM, MSCGM2 or the like; surgical adhesion preventive agents such as Interceed™, Seprafilm™ and the like; substrates such as Matrigel™, collagen, laminin and the like; growth factors such as VEGF, FGF2, HGF, EGF and the like; and cytokines such as IL6, IL1 beta and the like. Therefore, the pharmaceutical composition of the present invention may comprise these materials.

### EXAMPLE

Hereinbelow, the present invention will be described more specifically with reference to the following Example.

### Example 1

### Preparation of Hepatic Endodermal Cells (HE)

As for HE, human iPS cell-derived hepatic endodermal cells (Cell Reports 21, 2661-2670, 2017), PXB cells (PhoenixBio), etc. were used. To prepare a medium, GM BulletKit (Lonza) and HCM BulletKit (Lonza) from which hEGF (recombinant human epithelial cell growth factor) had been removed were mixed at a ratio of 1:1 and dexamethasone and Oncostatin M were added to the resulting mixture.

### Preparation of Mesenchymal Cells (MC)

As for MC, any of the following cells was used: cells isolated from human bone marrow (Lonza, cat. No. PT-2501), cells isolated from human umbilical cord stroma (Wharton's sheath), human iPS cell-derived mesenchymal cells (Cell Reports 21, 2661-2670, 2017), and the like. The mesenchymal stem cells isolated from human bone marrow (Mesenchymal Stem Cell: hMSC) that were mainly used in this experiment had been cultured using MSCGM2™ (Promocell C-28009), a medium prepared exclusively for hMSC culture.

### Preparation of Vascular Cells (EC)

As for EC, any of the following cells was used: human iPS cell-derived vascular endothelial cells (Cell Reports 21, 2661-2670, 2017), normal umbilical vein endothelial cells (HUVEC), and the like. With respect to HUVEC, either cells isolated from the umbilical cords provided, after informed consent, by maternal women at the time of delivery or purchased cells (HUVECs: Lonza, cat. No. 191027) were cultured in EGM™ BulletKit™ (Lonza CC-4133) through no more than 5 passages.

### Three-Type Cell Condensates

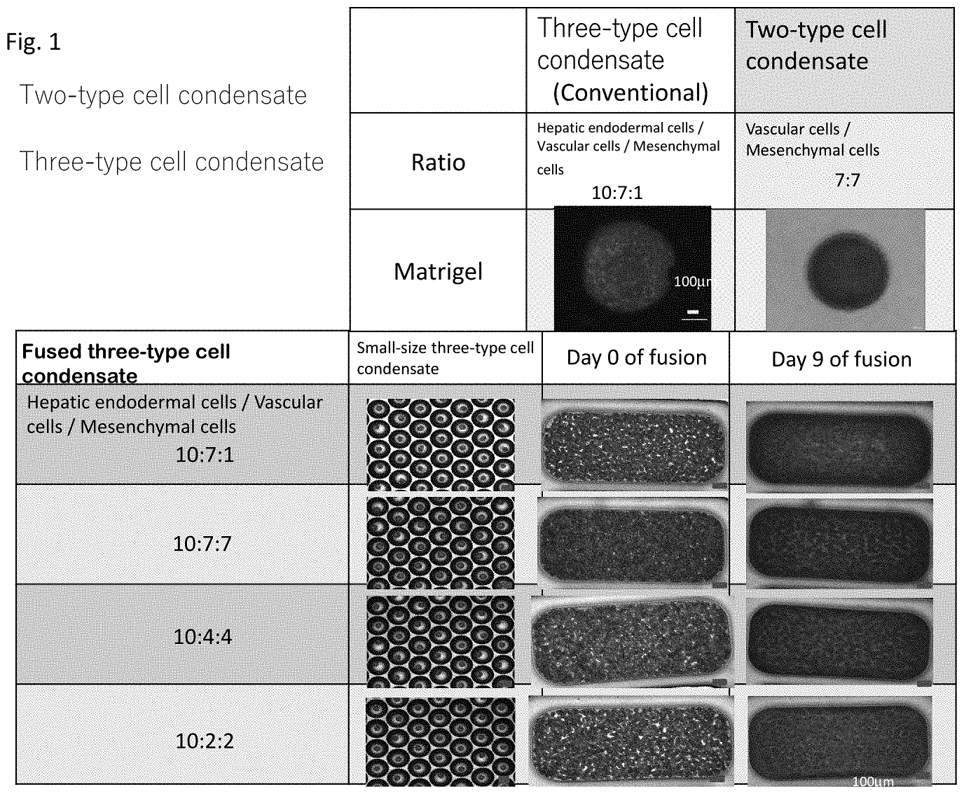

Stock solution of Matrigel coating (Corning™ Matrigel™) or a 1:1 mixed solution of Matrigel: medium was poured into 24-well plates (300 μl/well), which were then left stationary in a 37° C., 5% CO₂ incubator for 10 min or more for solidification. Then, 5×10⁵ cells of iPS cell-derived hepatic endodermal cells or human adult hepatocytes, 3.5×10⁵ cells of human iPS cell-derived vascular cells or human umbilical vein vascular endothelial cells, and 5×10⁴ cells of human iPS cell-derived mesenchymal cells or human mesenchymal cells were mixed in each well of 24-well plates, which were then incubated in a 37° C. incubator for two days. After seeding, observation over time of cell coculture was performed with a stereoscopic microscope. As shown in FIG. 1, formation of cell condensates was observed. It is also possible to prepare three-type cell condensates using micropatterned plates (FIG. 1). If desired, the three-type cell condensates prepared with micropatterned plates may be cultured on cell culture inserts (Falcon' Cell Culture Inserts) for approx. 1 to 9 days to prepare fused three-type cell condensates (FIG. 1).

### Two-Type Cell Condensates

Stock solution of Matrigel coating (Corning™ Matrigel™) or a 1:1 mixed solution of Matrigel: medium was poured into 24-well plates (300 μl/well), which were then left stationary in a 37° C., 5% CO₂ incubator for 10 min or more for solidification. Then, 5×10⁵ cells of human iPS cell-derived mesenchymal cells or human mesenchymal cells and 5×10⁵ cells of human iPS cell-derived vascular cells or human umbilical vein vascular endothelial cells were mixed in each well of 24-well plates, which were then incubated in a 37° C. incubator for two days. After seeding, observation over time of cell coculture was performed with a stereoscopic microscope. As shown in FIG. 1, formation of two-type cell condensates in the same manner as in three-type cell condensates was observed. It is also possible to prepare two-type cell condensates using micropatterned plates (FIG. 1).

### Expression of Fibrolysis Genes in Two-Type Cell Group

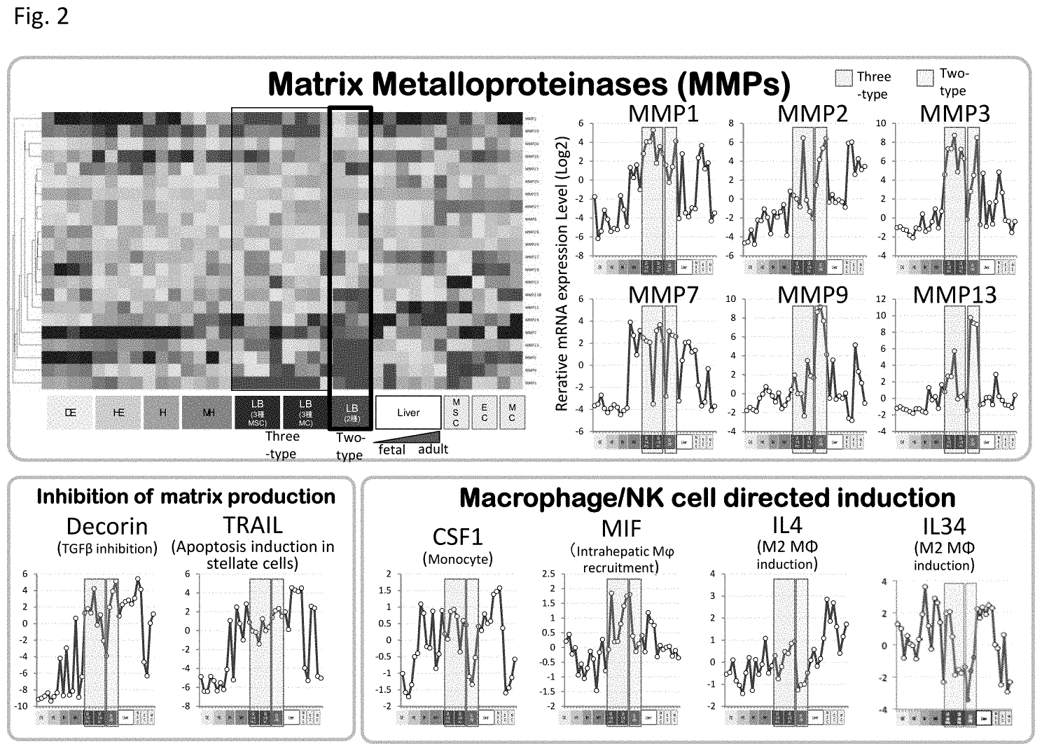

Comprehensive gene expression analysis data are shown for human iPS cell-derived hepatic endodermal cells (grouped by differentiation stage: DE, HE, IH and MH), three-type cell group, two-type cell group, hepatocytes (Liver), bone marrow-derived mesenchymal stem cells (MSC), human iPSC-derived vascular cells (EC), and human iPSC-derived mesenchymal cells (FIG. 2).

DE, IH and MH were prepared according to the method disclosed in Cell Reports 21, 2661-2670, 2017.

Comprehensive gene expression analysis was performed using SurePrint G3 Human GE Ver 2.0 (G4851B) (Agilent Technologies).

Relative mRNA expression levels were measured as described below. Briefly, array data were integrated on GeneSpring GX software and standardized with the 70% Shiftile method. Then, median correction was performed for each gene. The values obtained were plotted on the graph.

### Property Analysis of Three-Type Cell Condensates

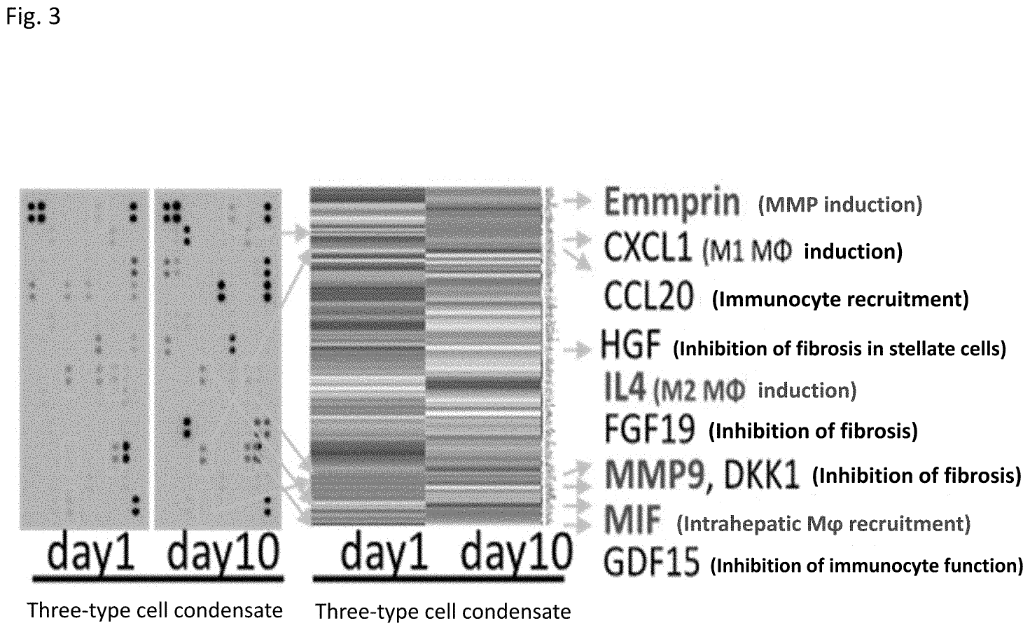

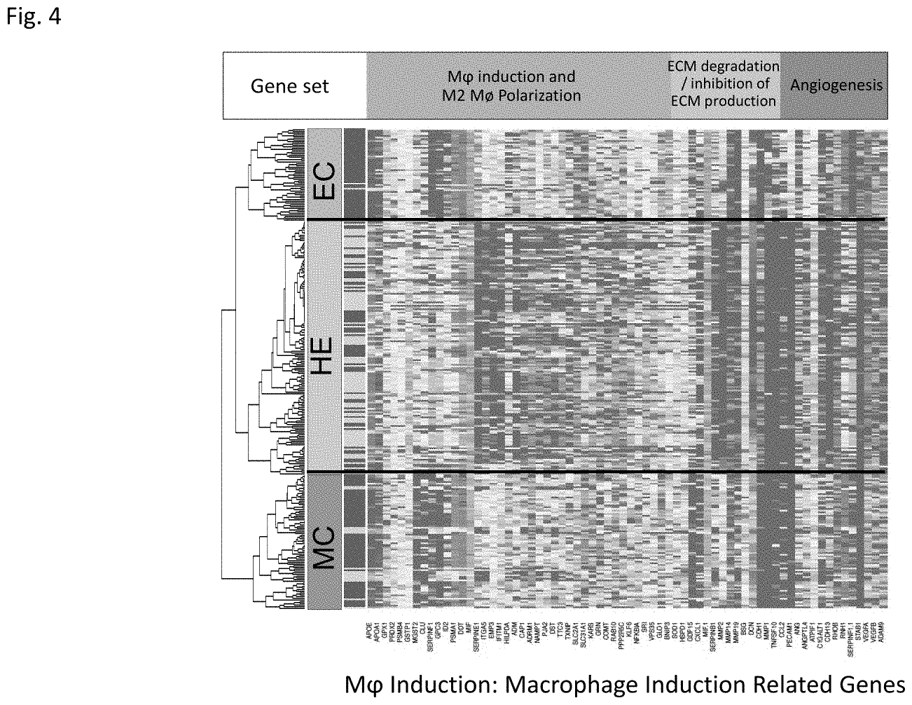

Comprehensive gene expression analysis data are shown for human iPS cell-derived hepatic endodermal cells (grouped by differentiation stage: DE, HE, IH and MH), three-type cell condensates and hepatocytes (Liver) (FIG. 2); cytokine array data are shown for the culture supernatant of three-type cell condensates (FIG. 3), and single cell RNA sequence analysis is also shown (FIG. 4).

As for cytokine array, Proteome Profiler Human XL Cytokine Array Kit (ARY022B) (R&D) was used. As for single cell RNA sequence analysis, cell isolation and RNA sample preparation were performed with C1 System (Fludigm), followed by analysis with a next-generation sequencer.

### Immunodeficient Mouse Liver Cirrhosis Model

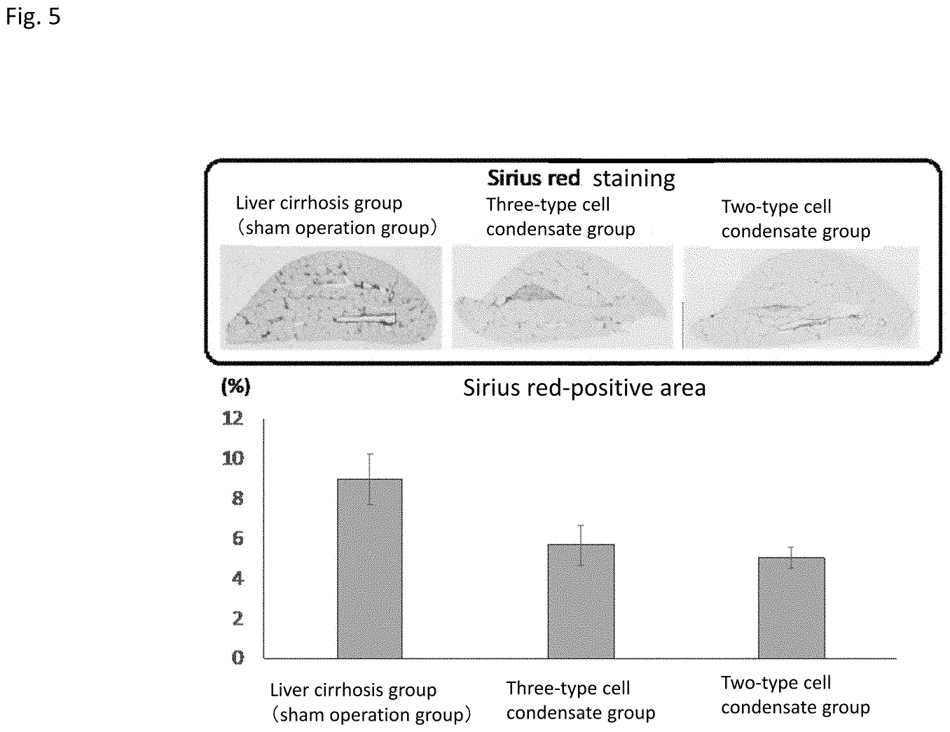

Thioacetamide (TAA) (WAKO) was injected intraperitoneally into immunodeficient mice (NOD/scid) at a concentration of 100 mg/kg body weight three times a week for two weeks to thereby prepare an immunodeficient mouse liver cirrhosis model. Subsequently, two-type and three-type cell condensates were transplanted onto the surface of the liver. From the day after the transplantation, TAA was administered for 2-10 weeks under the same conditions as used in the preparation of the cirrhosis model. Two weeks after the transplantation, histological analysis of engrafted tissue was performed and the therapeutic effects produced by two-type and three-type cell condensates were examined (FIGS. 5, 7, 8 and 10).

### Immunodeficient Rat Liver Cirrhosis Model

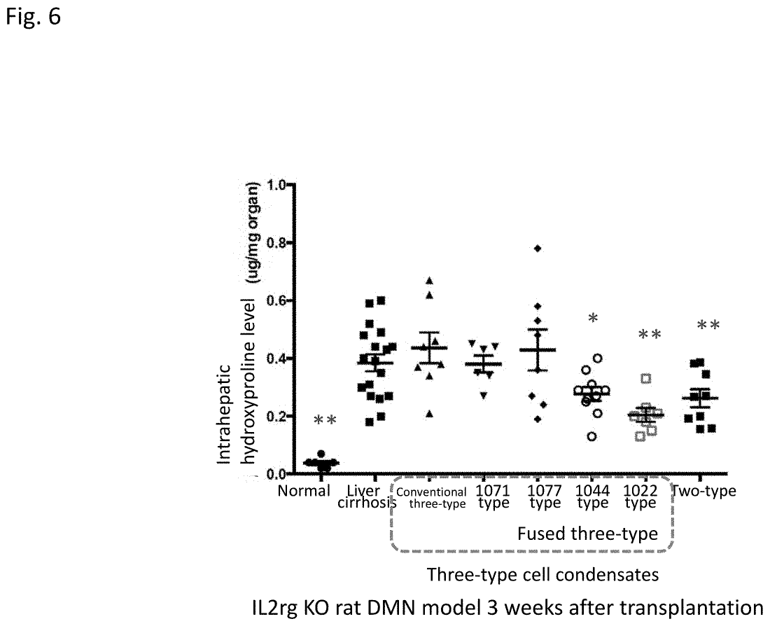

N-Nitrosodimethylamin (DMN) (WAKO) was injected intraperitoneally into immunodeficient rats (IL2rg KO) at a concentration of 10 mg/kg body weight three times a week for two weeks to thereby prepare an immunodeficient rat liver cirrhosis model. Subsequently, two-type and three-type cell condensates were transplanted onto the surface of the liver. From the day after the transplantation, DMN was administered for one week under the same conditions as used in the preparation of the cirrhosis model. Three weeks after the transplantation, histological analysis of engrafted tissue was performed and the therapeutic effects produced by two-type and three-type cell condensates were examined (FIGS. 6, 8, 9 and 11).

### Sirius Red Staining (FIG. 5)

Two-type and three-type cell condensates were transplanted into immunodeficient mice on the surface of the liver. Two weeks after the transplantation, liver tissues were resected and paraffin blocks were prepared. Thin sections were cut out from the paraffin blocks, treated with xylene for 10 min three times for deparaffinization, and rehydrated through a series of descending ethanol concentrations (50-100%). After replacing the ethanol solution with MilliQ water, the tissue sections were stained for 30 min with a Sirius red staining solution prepared by diluting 1% Sirius red solution (Muto Chemical) with saturated picric acid solution (Muto Chemical) to 0.03%. After washing, the tissue sections were dehydrated through a series of ascending ethanol concentrations (50-100%) and treated with xylene for 3 min three times for clearing. Mount-Quick (Daido Sangyo) was then added dropwise. Thereafter, the tissue sections were enclosed with a slide glass (Matsunami).

The upper panel of FIG. 5 shows the results of Sirius red staining of liver tissues two weeks after transplantation of three-type and two-type cell condensates into immunodeficient mouse liver cirrhosis model. The lower panel of FIG. 5 shows Sirius red-positive areas quantified using ImageJ (Rasband, W. S., ImageJ, U. S. National Institutes of Health, Bethesda, Maryland, USA, http://rsb.info.nih.gov/ij/, 1997-2012). Decrease in Sirius red-positive area was observed upon transplantation of three-type and two-type cell condensates.

### Collagen Levels (FIG. 6)

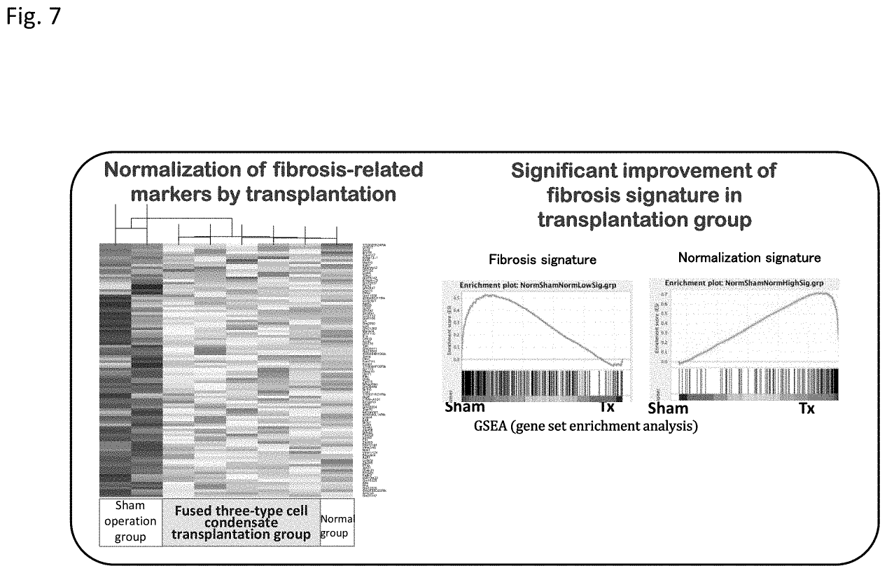

Part of the sampled organ was transferred into a BioMasher™ tube (Nippi Inc.). After adding 6N HCl (WAKO) in an amount four times the tissue weight, the sample was homogenized. The resultant sample was transferred into a tube equipped with a screw cap. After adding 6N HCl in an amount four times the sample weight, the tube was set in a heater and hydrolysis was carried out at 96° C. for 12-15 hr. The tube was taken out from the heater and cooled at room temperature. Subsequently, the tube was reversed and its content was homogenized and centrifuged at 15000 rpm for 5 min. An appropriate amount of the resultant sample was taken and 0.5 volumes of H₂O was added. To 20 μl of the sample, 75 μl of preliminarily prepared Chloramine T reagent [a mixture of 50% 2-propanol (WAKO), Chloramine T 3H₂O (SIGMA) and acetate-citrate buffer] was added and mixed. Subsequently, 75 μl of preliminarily prepared Ehrlich's reagent [a mixture of 2-propanol (WAKO), Dimethylaminobenzaldehyde (SIGMA) and perchloric acid (SIGMA)] was added. The resultant mixture was incubated at 60° C. for 10 min. Absorbance was measured at 560 nm, and the amount of collagen (hydroxyproline) was calculated.
RNA was extracted from livers of mouse liver cirrhosis model group, three-type cell condensate-transplanted group and healthy mouse group, and comprehensive gene expression analysis was performed (FIG. 7). For comprehensive gene expression analysis, Whole Mouse Genome Ver 2.0 4×44k (G4846A) (Agilent Technologies) was used. Thereafter, fibrosis associated factors were extracted.

### Histological Analysis of Engrafted Tissue

Thin paraffin sections were cut out from paraffin blocks of liver tissue, treated with xylene for 10 min three times for deparaffinization, and rehydrated through a series of descending ethanol concentrations (50-100%). The resultant sections, being soaked in citrate buffer, were autoclaved to activate antigens. After blocking with Protein Block, primary antibody was reacted overnight. After washing with 0.05% PBS-Tween, secondary antibody was reacted for an hour, followed by color development with DAB chromogen (DAKO). The following were used as the primary antibody: anti-human albumin antibody (Sigma), anti-human CD31 antibody (DAKO) and anti-human CK19 antibody (Dako).

### Blood Biochemical Data

Blood samples collected from liver cirrhosis model animals were centrifuged at 4000 rpm for 20 min, and sera were recovered. The recovered sera were analyzed for AST (aspartate aminotransferase), ALT (alanine aminotransferase), NH₃ (ammonia), ALB (albumin), T-Bil (total bilirubin) and D-Bil (direct bilirubin) using Fuji Dri-Chem slide (FujiFilm). For measurement, DRI-CHEM 7000V (FujiFilm) was used.

### Results

Results are shown in FIGS. 1 to 11.

Two-type and three-type cell condensates are shown (FIG. 1). Upper panel, left: an image of three-type cell condensate (liver bud) (hepatic endodermal cells:vascular cells:mesenchymal cells=10:7:1). Upper panel, right:images of two-type cell condensates (vascular cells:mesenchymal cells=7:1, 1:1 and 1:7). Equivalent morphologies are recognized between two-type and three-type cell condensates. Upper row shows cell condensates prepared with micropatterned plates. Bottom row shows cell condensates prepared with hydrogel. Vascular cells are stained with Kusabira Orange. Lower panel: images of fused three-type cell condensates (hepatic endodermal cells:vascular cells:mesenchymal cells=10:7:1, 10:7:7, 10:4:4 and 10:2:2). Microarray analysis of two-type and three-type cell condensate groups revealed that two-type cell condensate group was observed to have enhanced expression levels of fibrolysis enzymes, fibrogenesis inhibitor gene (TGF β inhibitor), differentiation-inducing factor for intrahepatic macrophages capable of higher yield expression of fibrolysis enzymes, etc. (FIG. 2). As a result of property analysis of three-type cell condensates, production of fibrolytic factors such as MMP1, MMP9, MMP13, Emmprin, etc. and cytokines such as HGF (inhibition of fibrosis in stellate cells), FGF19, MMP9, DKK1 (these three inhibit fibrosis), CXCL1 (M1 macrophage induction), IL4 (M2 macrophage induction), CCL20 (immunocyte recruitment), MIF (macrophage recruitment in the liver), GDF15 (inhibition of immunocyte function), etc. was confirmed (FIG. 3); and fibrosis inhibitory effect by production of these factors was presumed. Results of single cell RNA sequence analysis on hepatic endodermal cells, vascular cells and mesenchymal cells constituting three-type cell condensates are shown (FIG. 4). Mouse fetal liver-constituting cells (pink), three-type cell condensate-constituting cells (red). HE: hepatic endodermal cells; EC: vascular endothelial cells; MC: mesenchymal cells. Each of the three-type cell condensate-constituting cells is expressing the gene sets of “macrophage induction & M2 macrophage polarization”, “extracellular matrix (ECM) degradation and inhibition of ECM production” and “angiogenesis”, thus contributing to improvement of fibrosis.

FIG. 5 shows improvement of fibrosis by Sirius red staining two weeks after transplantation of three-type and two-type cell condensates into immunodeficient mouse liver cirrhosis model. As a result of transplantation of three-type and two-type cell condensates, decrease in Sirius red-positive area was observed.

FIG. 6 shows comparison of collagen levels in liver tissue three weeks after transplantation of three-type, two-type and fused three-type cell condensate into immunodeficient rat liver cirrhosis model. As regards fused three-type cell condensates, four mixing ratios were used for comparison; they were hepatic endodermal cells:vascular cells:mesenchymal cells=10:7:1, 10:7:7, 10:4:4 and 10:2:2. The collagen levels in the liver left lobe into which cell condensates were transplanted are shown. In fused three-type cell condensates (10:4:4 and 10:2:2) and two-type cell condensates, decrease in collagen level in liver tissue was observed.

According to the microarray data on liver tissues from three groups, i.e. normal group, liver cirrhosis group (sham operation group) and three-type cell condensate transplantation group, decrease in fibrosis signature and increase in normalization signature were observed in the transplantation group upon transplantation of three-type cell condensate (FIG. 7).

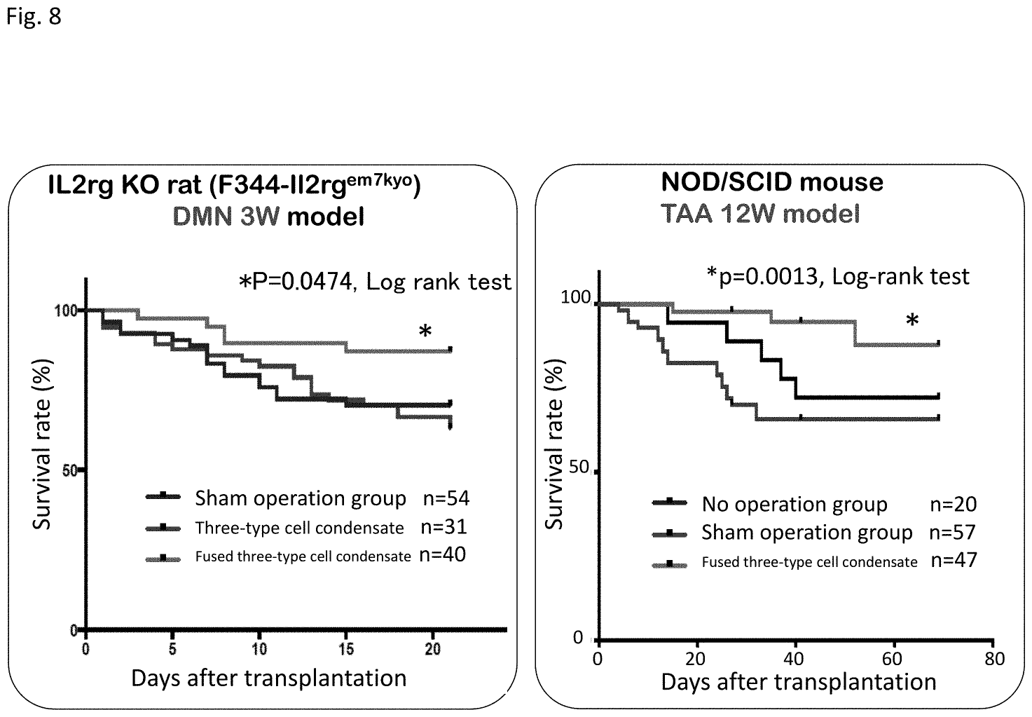

FIG. 8 shows survival rates after transplantation of fused three-type cell condensates into immunodeficient rat and mouse liver cirrhosis models. Survival rates up to 3 weeks (for rat) and 10 weeks (for mouse) after transplantation are shown. Left panel (rat): the survival rate of fused three-type cell condensate transplantation group was significantly improved compared to sham operation group and three-type cell condensate transplantation group. Right panel (mouse): the survival rate of fused three-type cell condensate transplantation group was significantly improved compared to sham operation group and no operation group.

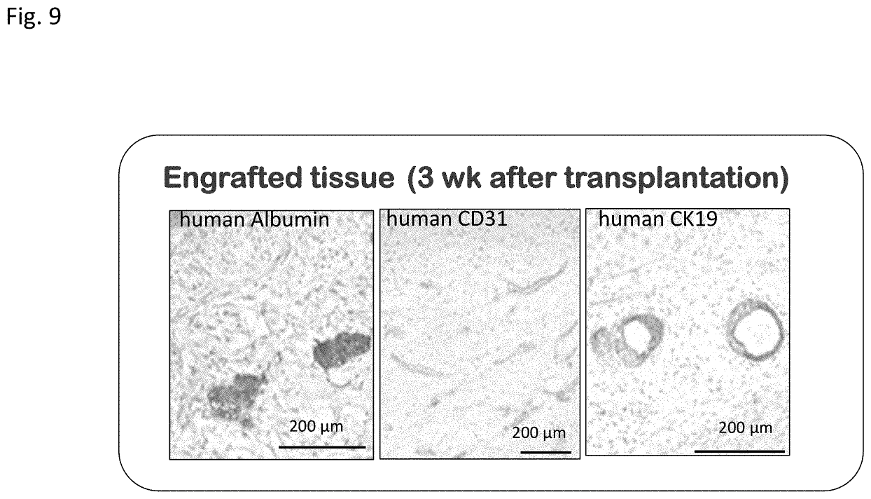

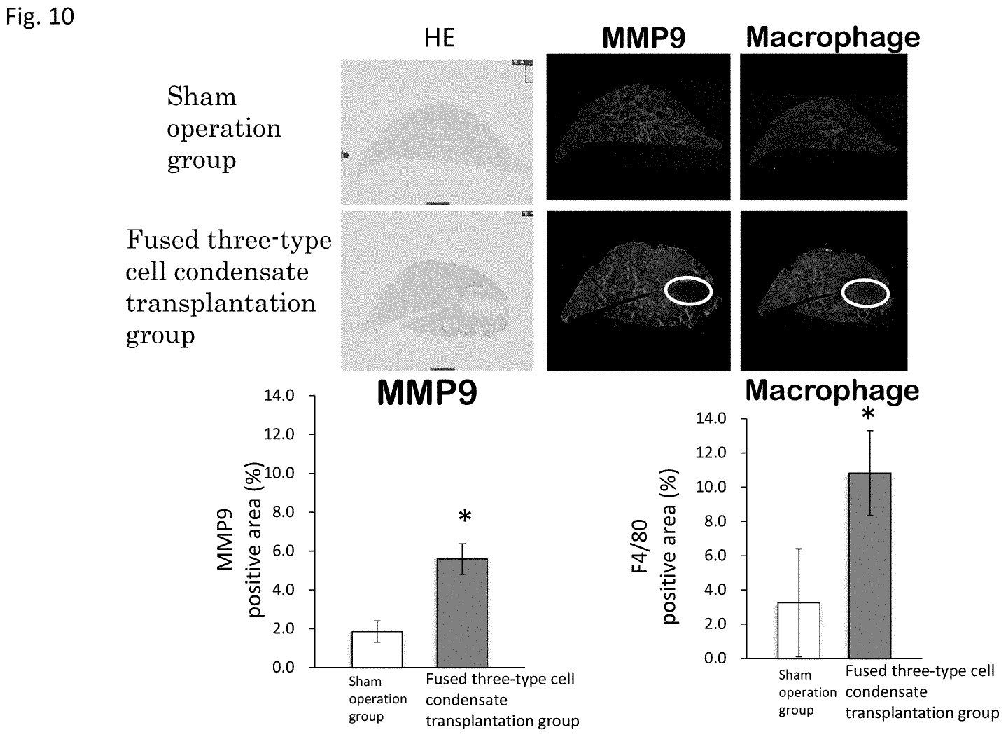

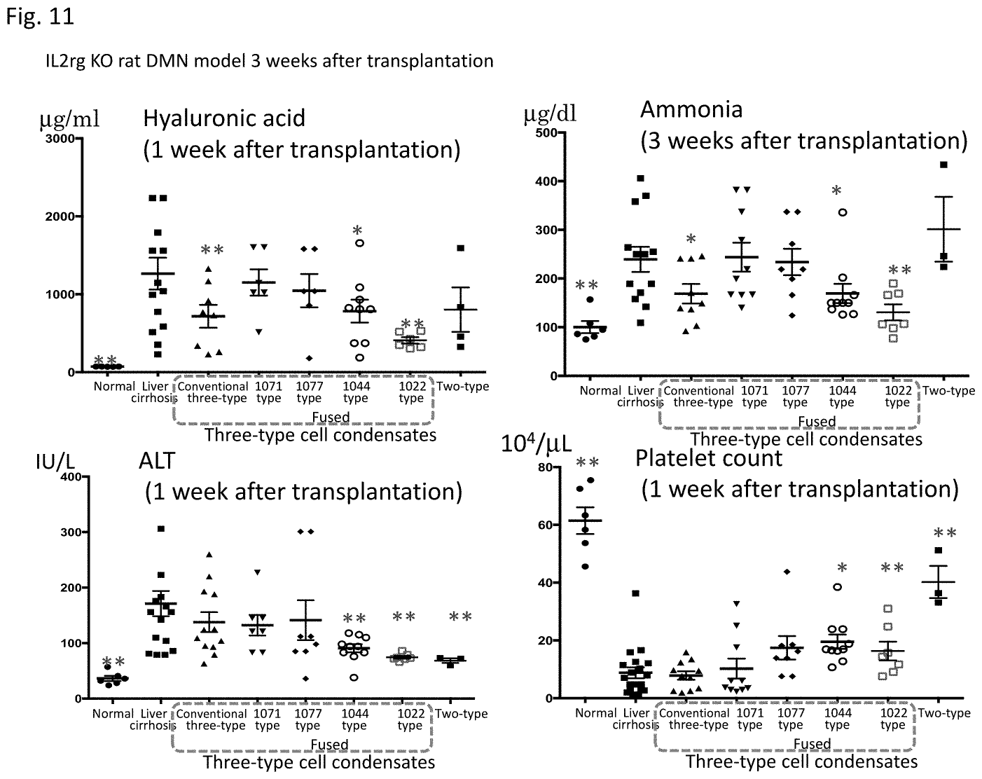

Upon close examination of images of engrafted tissues three weeks after transplantation of fused three-type cell condensates into immunodeficient rat liver cirrhosis model, human albumin-positive hepatocytes, human CD31-positive blood vessel-like structures, and human CK19-positive bile duct structures were observed (FIG. 9). FIG. 10 shows images of macrophage accumulation and MMP9 accumulation six weeks after transplantation of fused three-type cell condensates into immunodeficient mouse liver cirrhosis model. Compared to sham operation group, fused three-type cell condensate transplantation group was observed to experience a significant increase in macrophage accumulation and MMP9 secretion. FIG. 11 shows biochemical data on the blood from 1 to 3 weeks after transplantation of two-type and three-type cell condensates into immunodeficient rat liver cirrhosis model. A tendency toward improvement was observed in hyaluronic acid, NH3, ALT and platelets as a result of transplantation.

In conclusion, cell condensates of two types of cells (mesenchymal stem cells and vascular endothelial cells) or three types of cells (hepatic endodermal cells, mesenchymal cells and vascular cells) according to the present invention are engrafted in liver tissue for a prolonged period over which fibrolytic enzymes are continuously produced. Therefore, it is possible to inhibit hepatic fibrosis efficiently and effectively. An equivalent effect can also be expected in cell mixtures of two types of cells (mesenchymal stem cells and vascular endothelial cells) or three types of cells (hepatic endodermal cells, mesenchymal cells and vascular cells). Predictably, fibrosis will be improved by transplanting such cell mixtures and binding them in the body. Moreover, a remarkable fibrosis improvement effect was observed even in two-type cell condensates (mesenchymal cells and vascular cells) that did not contain a major organ or tissue cell such as hepatocyte. Therefore, inhibition of fibrosis can be expected in various organs and/or tissues including the liver.

All publications, patents and patent applications cited herein are incorporated herein by reference in their entirety.

### INDUSTRIAL APPLICABILITY

The present invention is applicable to prevention and/or treatment of diseases accompanied by fibrosis in an organ and/or a tissue.

1. A pharmaceutical composition for preventing and/or treating a disease accompanied by fibrosis in an organ and/or a tissue, which comprises a cell mixture and/or a cell condensate comprising mesenchymal cells and vascular cells.

2. The composition of claim 1, wherein the ratio of mesenchymal cells to vascular cells in the cell mixture and/or the cell condensate is 1-10:10-1.

3. The composition of claim 1, wherein the cell condensate comprising mesenchymal cells and vascular cells has been prepared by coculturing mesenchymal cells and vascular cells.

4. The composition of claim 1, wherein the mesenchymal cell is undifferentiated.

5. The composition of claim 1, wherein the vascular cell is vascular endothelial cells.

6. The composition of claim 1, wherein the mesenchymal cell is derived from ES cells or iPS cells.

7. The composition of claim 1, wherein the vascular cell is derived from ES cells or iPS cells.

8. The composition of claim 1, wherein the cell mixture and/or the cell condensate further comprises hepatocytes.

9. The composition of claim 8, wherein the ratio of hepatocytes, mesenchymal cells and vascular cells in the cell mixture and/or the cell condensate is 10:0.1-10:0.1-10.

10. The composition of claim 8, wherein the cell condensate comprising hepatocytes, mesenchymal cells and vascular cells has been prepared by coculturing hepatocytes, mesenchymal cells and vascular cells.

11. The composition of claim 8, wherein the hepatocyte is hepatic endodermal cells.

12. The composition of claim 8, wherein the hepatocyte is derived from ES cells or iPS cells.

13-15. (canceled)

16. A method of preventing and/or treating a disease accompanied by fibrosis in an organ and/or a tissue, which comprises transplanting into a subject a pharmaceutically effective amount of a cell mixture and/or a cell condensate comprising mesenchymal cells and vascular cells.

17. The method of claim 16, wherein the cell mixture and/or the cell condensate further comprises hepatocytes.

18. A method of inhibiting fibrosis in an organ and/or a tissue, which comprises transplanting into a subject a pharmaceutically effective amount of a cell mixture and/or a cell condensate comprising mesenchymal cells and vascular cells.

19. The method of claim 18, wherein the cell mixture and/or the cell condensate further comprises hepatocytes.

20-25. (canceled)
